(19) United States
(12) Patent Application Publication
(10) Pub. No.: US 2008/0033567 A1
(43) Pub. Date: Feb. 7, 2008

(54) Knee joint prosthesis used in total knee arthroplasty

(76) Inventor: Robert P. Stchur, Punta Gorda, FL (US)

Correspondence Address: EDWARD M. LIVINGSTON, PA, 963 TRAIL TERRACE DRIVE, NAPLES, FL, 34103, US

(21) Appl. No.: 11/498,560

(22) Filed: Aug. 3, 2006

(51) Int. Cl. A61F 2/38

(52) U.S. Cl. 623/20.33

(57) ABSTRACT

A knee joint prosthesis used in total knee arthroplasty having tibial components (5) and tibial bearing inserts (20) with predetermined shapes that allow for an additional degree of freedom in both an anterior-posterior direction and rotation. The tibial bearing inserts (20) include a predeterminedly-shaped extension (17), such as a sphere (8), disc (12) or trapezoid (13), that extends downwardly from the bearing (4). The tibial components (5) include a socket (9) having a corresponding predetermined shape of the extension (17) and retaining edges (10) located at a top of the socket (9) so the extension (17) may be “dropped into” the socket (9) and therein retained. In this manner, disengagement of the tibial bearing inserts (20) at extremes of anterior or posterior translation of the bearing relative to the tibial component (5) is prevented without affected the translation and rotation of the tibial component (5) and tibial bearing inserts (20).

### BACKGROUND OF THE INVENTION

This invention relates to a knee joint prosthesis, more particularly, a knee joint prosthesis used in total knee arthroplasty having tibial bearing inserts and tibial components that provide both axial rotation and anterior-posterior rotation of the prosthetic knee as compared to the conventional fixed bearing knees.

A knee joint is created when a top of the shinbone, called the tibia, and a bottom of a thighbone, called a femur,join together. A person's kneecap, orpatella, is anteriorto the tibia and femur and cartilage provides padding between the bones to assist in smooth, gliding movement of the knee joint. A synovial membrane produces a lubricant to further assist in the smooth, gliding movement of the knee joint. However, arthritis, trauma to the knee joint or excessive stress placed on the knee joint over time breaks down the knee joint, making movement painful, difficult and sometimes impossible.

To help a patient regain painless movement of the knee joint, total knee arthroplasty was created and has since revolutionized the treatment of knee osteoarthritis. Total knee arthroplasty comprises implanting a prosthetic knee having a femoral component, a tibial component (also known as a “tibial tray”), a bearing component (also known as a “tibial insert”) and a patella into a patient so as to replace a bad knee joint. The stability of the prosthetic knee is dictated by the tightness of the ligaments between the femoral component and the tibial component.

The femoral component is typically made of metal and is attached to lower end of the femur so as to cover the lower end of the femur. The tibial component, which is secured in the tibia, typically has an insert having a metal base and a cushion, which is also known as a bearing, that is inserted into the tibial component. The bearing allows for a smooth, gliding surface between the femoral component and the tibial component. Finally, the patella is typically made of plastic, such as polyethylene, or of a combination of metal and plastic.

In its early design, the knee prosthesis acted as a true hinge joint with motion possible only in the sagittal plane. Known as a fixed bearing design wherein the bearing is locked into the tibial component by using bone cement, the fixed bearing design led to high rates of loosening due to the amount of stress transmitted to the bone-cement or bone-prosthesis interface.

Subsequent unconstrained designed dramatically increased the survivorship of the prosthesis. However, even with the traditional unconstrained design, the prosthesis tends to have a limited lifetime before loosening, especially on the tibial component. This, in turn, caused surgeons to limit the total knee arthroplasty procedure to those over sixty years of age. However, as there are a large number of individuals needing total knee arthroplasty under the age of sixty, the need for an even more unconstrained yet stable knee became of great importance.

Since then, several prosthetic knee designs have lessened the amount of force imparted on the bone-prosthesis interface by increasing the degree of freedom capable by the prosthesis. The first was a mobile bearing knee which allowed additional anterior-posterior movement of the bearing wherein the tibial component is topped with a flat element that holds the bearing in place. The second design included a rotating platform to allow rotational freedom wherein the tibial component is topped with a disk-shaped bearing that sits on a surface and rotates about a conical post. Although each design did increase freedom by one degree and retained stability to the knee, there is room for improvement.

The relevant prior art includes the following references:

| U.S. Pat. No. | | |
|---|---|---|
| (U.S. unless stated otherwise) | Inventor | Issue/Publication Date |
| 6,623,526 | Lloyd | Sep. 23, 2003 |
| 6,045,581 | Burkinshaw | Apr. 04, 2000 |
| 5,871,543 | Hofmann | Feb. 16, 1999 |
| 4,309,778 | Buechel et al. | Jan. 12, 1982 |
| 5,395,401 | Bahler | Mar. 07, 1995 |
| 4,470,158 | Pappas et al. | Sep. 11, 1984 |
| 2004/0030398 | Ferree | Feb. 12, 2004 |
| 6,875,235 | Ferree | Apr. 05, 2005 |
| 2004/0068322 | Ferree | Apr. 08, 2004 |
| 6,709,461 | O'Neil et al. | Mar. 23, 2004 |
| 6,443,991 | Running | Sep. 03, 2002 |
| 6,726,723 | Running | Apr. 27, 2004 |
| 2005/0192672 | Wyss et al. | Sep. 01, 2005 |
| 2004/0162620 | Wyss | Aug. 19, 2004 |
| 6,926,738 | Wyss | Aug. 09, 2005 |
| 6,660,039 | Evans et al. | Dec. 09, 2003 |
| 6,962,607 | Gundlapalli et al. | Nov. 08, 2005 |
| 6,616,696 | Merchant | Sep. 09, 2003 |
| 6,645,251 | Salehi et al. | Nov. 11, 2003 |
| 6,506,216 | McCue et al. | Jan. 14, 2003 |
| 6,869,447 | Lee et al. | Mar. 22, 2005 |
| WO01/13825 | Walker | Mar. 01, 2001 |

### SUMMARY OF THE INVENTION

The primary object of the present invention is to provide tibial bearing inserts and tibial components to be used in total knee arthroplasty that provide both axial rotation and anterior-posterior rotation of the prosthetic knee.

A further object of the present invention is to provide tibial bearing inserts and tibial components to be used in total knee arthroplasty that impart two additional degrees of freedom to the prosthetic knee.

An even further object of the present invention is to provide tibial bearing inserts and tibial components to be used in total knee arthroplasty that reduce the amount of stress imparted on the bone-cement or bone-prosthesis interface.

Another object of the present invention is to provide tibial bearing inserts and tibial components to be used in total knee arthroplasty that significantly increase the survivorship of the knee prosthesis.

An even further object of the present invention is to provide tibial bearing inserts and tibial components to be used in total knee arthroplasty that do not sacrifice knee stability.

A further object of the present invention is to provide tibial bearing inserts and tibial components to be used in total knee arthroplasty could allow for greater flexion.

The present invention fulfills the above and other objects by providing tibial bearing inserts and tibial components having predetermined corresponding shapes that allow for an additional degree of freedom in both an anterior-posterior direction and rotation. The tibial bearing inserts include a predeterminedly-shaped extension, such as a sphere, disk or trapezoid, that extends downwardly from the bearing. The tibial components include a socket having a corresponding predetermined shape of the extension and retaining edges located at a top of the socket so the extension may be “dropped into” the socket and therein retained. In this manner, disengagement of the tibial bearing insert at extremes of anterior or posterior translation of the bearing relative to the tibial component is prevented without affected the translation and rotation of the tibial component and tibial bearing insert.

The prosthetic knee can either be posterior stabilized or cruciate retaining.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1A is a perspective view of an operable prosthetic knee of the prior art;

FIG. 1B is a perspective view of the various components of a prosthetic knee of the prior art;

FIG. 2 is a perspective view of a first embodiment of a tibial component and bearing of the present invention;

FIG. 3 is a top plan view of a base of the first embodiment of a tibial component of the present invention;

FIG. 4 is a side sectional view of the first embodiment of a tibial component and tibial bearing insert of the present invention;

FIG. 5 is a sectional view of the first embodiment of a tibial component and tibial bearing insert in an anterior position of the present invention;

FIG. 6 is a sectional view of the first embodiment of a tibial component and tibial bearing insert in a central position of the present invention;

FIG. 7 is a sectional view of the first embodiment of a tibial component and tibial bearing insert in a posterior position of the present invention;

FIG. 8 is a top view of a bearing of the present invention during axial rotation;

FIG. 9 is a side sectional view of the first embodiment of a tibial component and tibial bearing insert in a flexion position of the present invention;

FIG. 10 is a side sectional view of the first embodiment of a tibial component and tibial bearing insert in an extension position of the present invention;

FIG. 11 is a perspective view of a second embodiment of a tibial component and tibial bearing insert of the present invention;

FIG. 12 is a sectional view of the second embodiment of a tibial component and tibial bearing insert in an anterior position of the present invention;

FIG. 13 is a sectional view of the second embodiment of a tibial component and tibial bearing insert in a central position of the present invention;

FIG. 14 is a top plan view of a base of the second embodiment of a tibial component of the present invention;

FIG. 15 is a sectional view of a third embodiment of a tibial component and tibial bearing insert in an anterior position of the present invention;

FIG. 16 is a sectional view of the third embodiment of a tibial component and tibial bearing insert in a central position of the present invention;

FIG. 17 is a top plan view of a base of the third embodiment of a tibial component of the present invention;

FIG. 18 is a sectional view of a fourth embodiment of a tibial component and tibial bearing insert in an anterior position of the present invention;

FIG. 19 is a sectional view of the fourth embodiment of a tibial component and tibial bearing insert in a central position of the present invention;

FIG. 20 is a sectional view of a fifth embodiment of a tibial component and tibial bearing insert in an anterior position of the present invention;

FIG. 21 is a sectional view of the fifth embodiment of a tibial component and tibial bearing insert in a central position of the present invention;

FIG. 22 is a sectional view of a sixth embodiment of a tibial component and tibial bearing insert in a central position of the present invention; and

FIG. 23 is a sectional view of the sixth embodiment of a tibial component and tibial bearing insert in a central position of the present invention.

### DESCRIPTION OF THE PREFERRED EMBODIMENTS

For purposes of describing the preferred embodiment, the terminology used in reference to the numbered components in the drawings is as follows:
1. femur
2. tibia
3. femoral component
4. bearing
5. tibial component
6. patellar component
7. tibial component base plate
8. sphere
9. socket
10. retaining edge
11. track
12. disk
13. trapezoid
14. central socket area
15. patella
16. cone
17. extension
18. perimeter
19. bearing base plate
30. tribial bearing insert

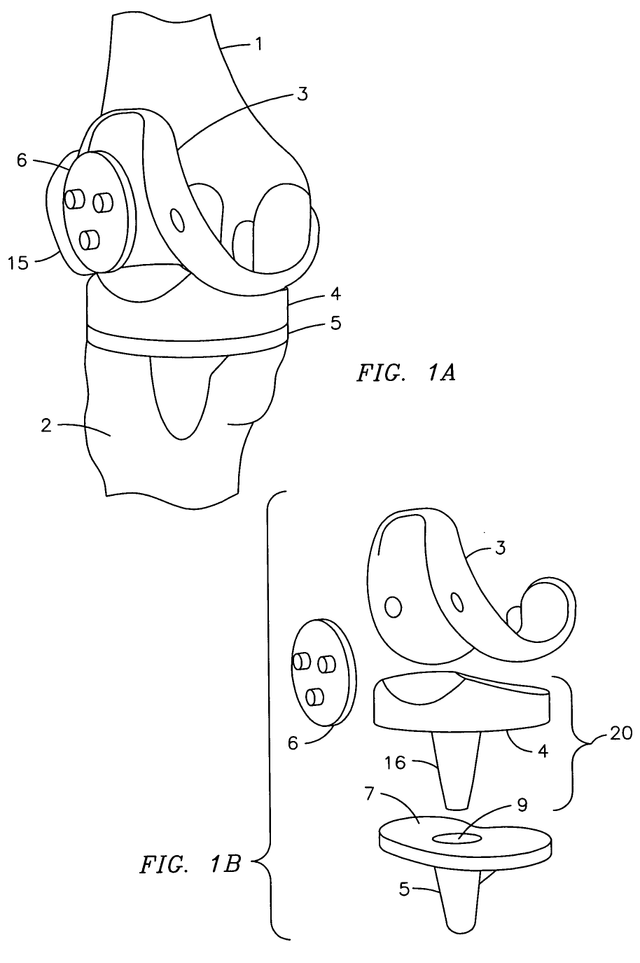

Referring to FIGS. 1A and 1B, the prior art components currently used in total knee arthroplasty are shown. During total knee arthroplasty, also referred to as knee replacement surgery, a patient's knee is replaced with a prosthetic knee. After entering the knee joint via an incision, a surgeon utilizes a cutting jig to shape the distal end of the femur 1 so as to cut the femur 1 in proper alignment to the leg's original angles. Then, the proximal end of the tibia 2 is also cut so as to be in proper alignment. The undersurface of the patella 15, also referred to as a kneecap, is then removed. A femoral component 3, which is made of metal, is then placed on the femur 1 and is secured thereto by a taper on the end of the bone or by utilizing bone cement. Then, a tibial component 5 having a tibial component base plate 7 is attached to the proximal end of the tibia 2. The tibial component base plate 7 is secured to the tibial component 5 via bone cement or screws. A tibial bearing insert 20, having a bearing 4 that is typically made of polyethylene and a downwardly extending cone 16, is then inserted into the tibial component 5 such that the downwardly extending cone 16 is inserted into a socket 9 of the tibial component 5. Finally, a patellar component 6 is cemented behind the patella 15 and the incision is closed. Although the prosthetic knee currently used in total knee arthroplasty provides a working knee joint, oftentimes the bearing 4 will need replacement over a period of time due to wear, which requires additional surgeries. In addition, the prosthetic knee joint is limited in the degrees of movement as compared to a healthy knee.

On the other hand, the present invention permits a longer lasting prosthetic knee having a greater range of movement by providing improved tibial components and bearings that are used in conjunction with a femoral component and patellar component for total knee arthroplasty. As shown in FIG. 2, a first embodiment of the present invention shows a perspective view of a tibial component and tibial bearing insert of the present invention. The tibial component 5 is preferably made of a porous material so as to promote ingrowth; however, the tibial component may also be smooth as well. In addition, the tibial component base plate 7 is preferably made of a highly polished metal, although other material, such as polyethylene, may also be used. Similar to the convention tibial component 5, the tibial component 5 of the present invention includes a socket 9.

However, unlike the tibial bearing insert 20 of the prior art, the tibial bearing insert 20 of a first embodiment of the present invention includes a extension 17 of a predetermined shape and a predetermined perimeter 18, in this embodiment a ball or sphere 8. The sphere 8 is preferably highly polished metal but may also be polyethylene. The sphere 8 is then inserted into the socket 9.

In FIG. 3, a top plan view of the tibial component base plate 7 of a first embodiment of the tibial component 5 of the present invention is shown. The tibial component base plate 7 includes a socket 9 having a predetermined shape for acceptance of the extension 17 of the tibial bearing insert 20. A central socket area 14 of the socket 9 has corresponding angles equal and corresponding line segments proportional to the greatest perimeter 18 of the predetermined shape of the extension 17. Thus, the central socket area 14 is similar to the perimeter 18 of the extension 17. In the first embodiment, the perimeter 18 of the sphere 8, which is commonly referred to as the circumference, is slightly smaller than the central socket area 14, thereby allowing the sphere 8 to be easily inserted into the central socket area 14. Once within the socket 9, the extension 17 is movable within and along a track 11 having a predetermined shape and size that corresponds to the shape of and is slightly larger than the extension 17, thereby permitting anterior A and posterior P movement of the tibial bearing insert 20. Thus, the track 11 is key significance in assisting the mobility and rotation of the tibial bearing insert 20 as the prior art only permitted rotational movement of the tibial bearing insert 20 due to the socket not permitting lineal movement once the tibial bearing insert 20 was inserted into the tibial component 5. In addition, rotational movement of the bearing 4 is also permitted when the extension 17 is located within the socket 9. Moreover, because the socket 9 includes at least one retaining edge 10 that covers a predetermined portion of the track 11, the extension 17 is retained within the socket 9. Thus, only when the extension 17 is located directly beneath the central socket area 14 in its entirety may the extension 17 be removed from the socket 9.

In FIG. 4, a side sectional view of the first embodiment of a tibial component and bearing of the present invention is shown. The tibial bearing insert 20 includes a sphere 8, which is preferably secured to a bearing base plate 19 which may be metal, polyethylene or any other material, that is placed into the socket 9 of the tibial component 5. The retaining edges 10 of the tibial component 5 cover a predetermined amount of the track 11 so as to assist in retaining the sphere 8 within the socket 9 and along the track 11.

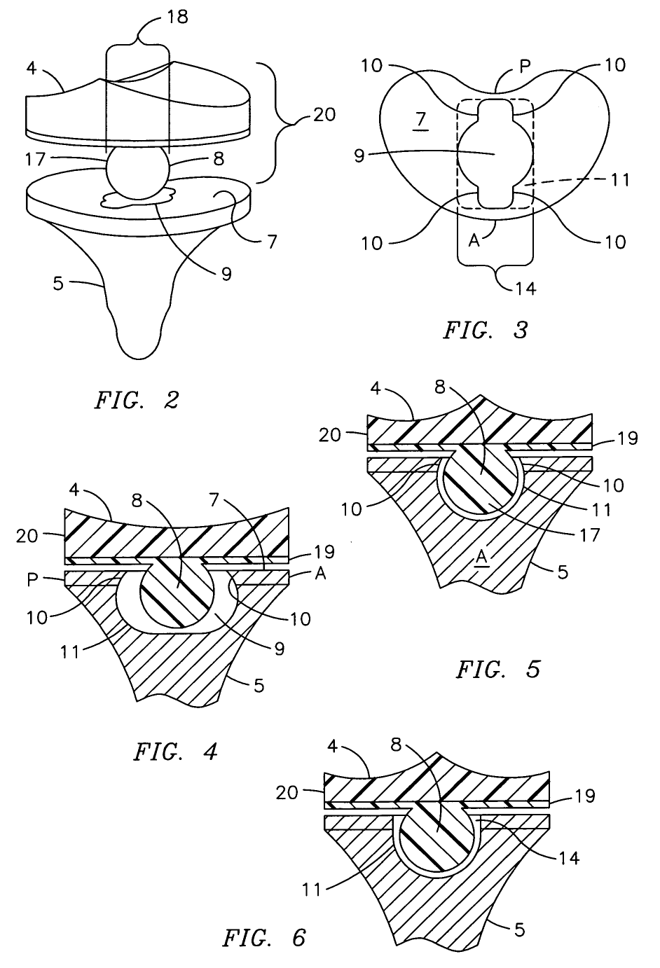

In FIG. 5, a sectional view of the first embodiment of a tibial component and bearing in an anterior position of the present invention is shown. When in an anterior A position, the extension 17, or as in the first embodiment, sphere 8, is locked within the socket 9 by the retaining edges 10.

On the other hand, when the sphere 8 is located within the central socket area 14 as shown in FIG. 6, the sphere 8 is able to be removed as there are no retaining edges 10 to retain the sphere within the socket 9.

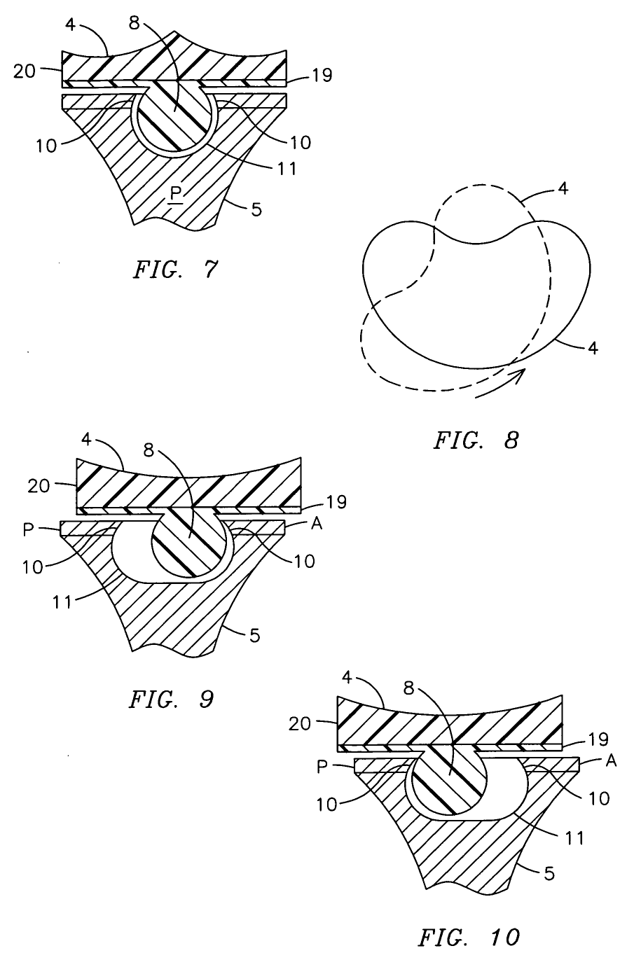

When the sphere is located in a posterior P position within the socket 9, as shown in FIG. 7, the sphere is once again locked within the socket 9 by the retaining edges 9. The inclusion of the retaining edges 10 on the socket 9 is of great importance as it not only retains the extension 17 within the socket 9, but is also permits the extension 17, and thus bearing 4, to have anterior-posterior translation and rotation.

In FIG. 8, a top view of a tibial bearing insert of the present invention during axial rotation is shown. The tibial bearing insert 20 is able to rotate axially because of the design of the extension 17 and corresponding socket 9 having at least one retaining edge 10.

In FIG. 9, a side sectional view of the first embodiment of a tibial component and tibial bearing insert in a flexion position of the present invention is shown. During flexion, tibial bearing insert 20 extends partially over the anterior A side of the tibial component 5 so as to permit bending of the prosthetic knee.

In FIG. 10, a side sectional view of the first embodiment of a tibial component and tibial bearing insert in an extension position of the present invention is shown. During extension, the tibial bearing insert 20 partially extends over the posterior P side of the tibial component 5 so as to permit straightening of the prosthetic knee.

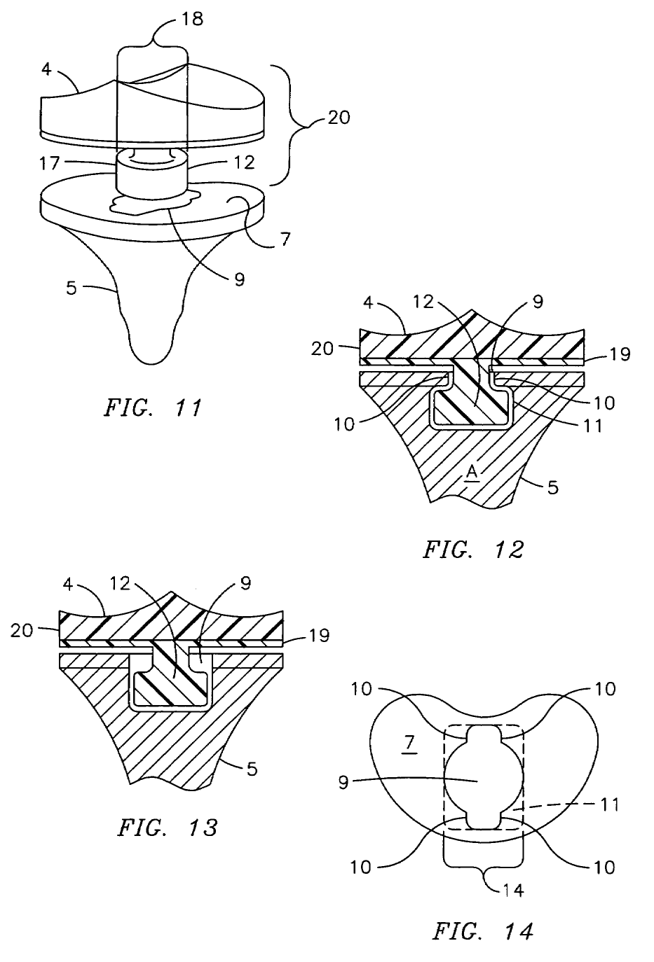

Referring to FIGS. 11-14, varying views of a second embodiment of the tibial component 5 and tibial bearing insert 20 of the present invention are shown. Similar to the first embodiment, the second embodiment of the present invention includes an extension 17 having a predetermined shape with a perimeter 18, a socket 9 having a central socket area 14 having a similar perimeter 18 as the extension 17, at least one retaining edge 10 that covers a predetermined portion of a track 11 wherein the track 11 is predeterminedly shaped and sized so as to permit movement of the extension 17. However, in the second embodiment, the extension 17 is in the shape of a disc 12. Thus, the socket 9 located in the tibial component 5 has a corresponding disc shape so as to accept the disc 12 via the central socket area 14. The track 11 also has a predetermined shape that permits movement of the disc 12 is an anterior-posterior manner, as well as permitting the disc 12, and thus bearing 4, rotational movement.

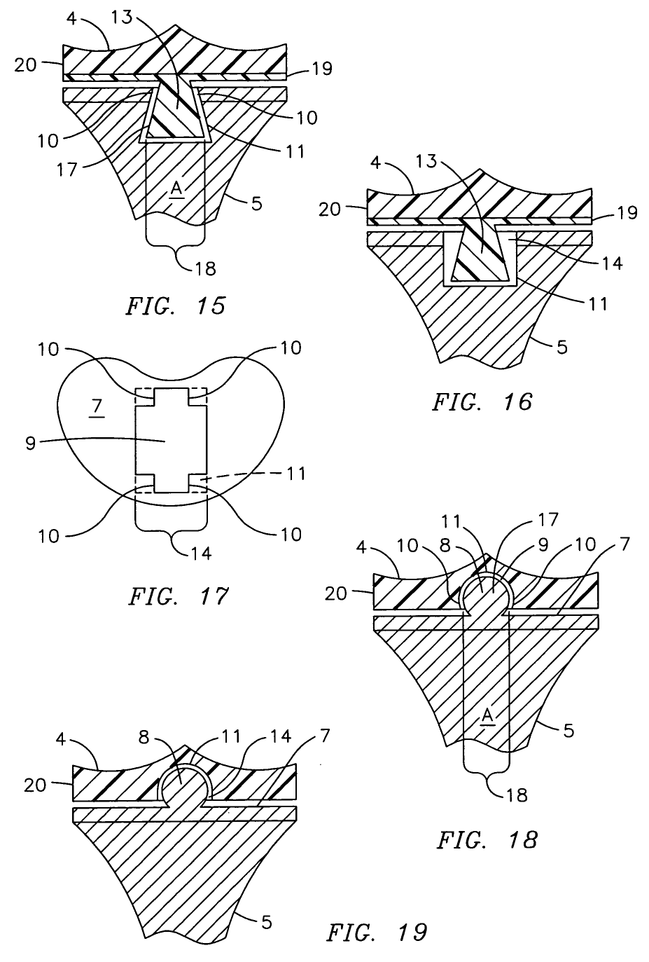

Next, in FIGS. 15-17, varying views of a third embodiment of the tibial component 5 and tibial bearing insert 20 of the present invention are shown, the only difference being the shape of the extension 17 being a trapezoid 13 and the central socket area 14 and track 11 being shaped and sized for acceptance of same.

In FIGS. 18 and 19, varying sectional views of a fourth embodiment of a tibial component and bearing in an anterior position of the present invention is shown. Similar to the previous embodiments of the present invention, the fourth embodiment includes an extension 17 having a predetermined shape and perimeter 18, a socket 9 having a central socket area 14 that is similar to the perimeter 18 of the extension 17, at least one retaining edge 10 covering a predetermined portion of the track 11 and a track 11 sized and shaped for movement of the extension 17. However, in the fourth embodiment, the extension 17, in this case sphere 8, is located on the tibial component 5, preferably attached to the tibial component base plate 7, and the socket 9 is located on the tibial bearing insert 20. Although the location of the extension 17 and socket 9 are essentially reversed, anterior-posterior and rotational movement of the tibial bearing insert 20 is still permitted.

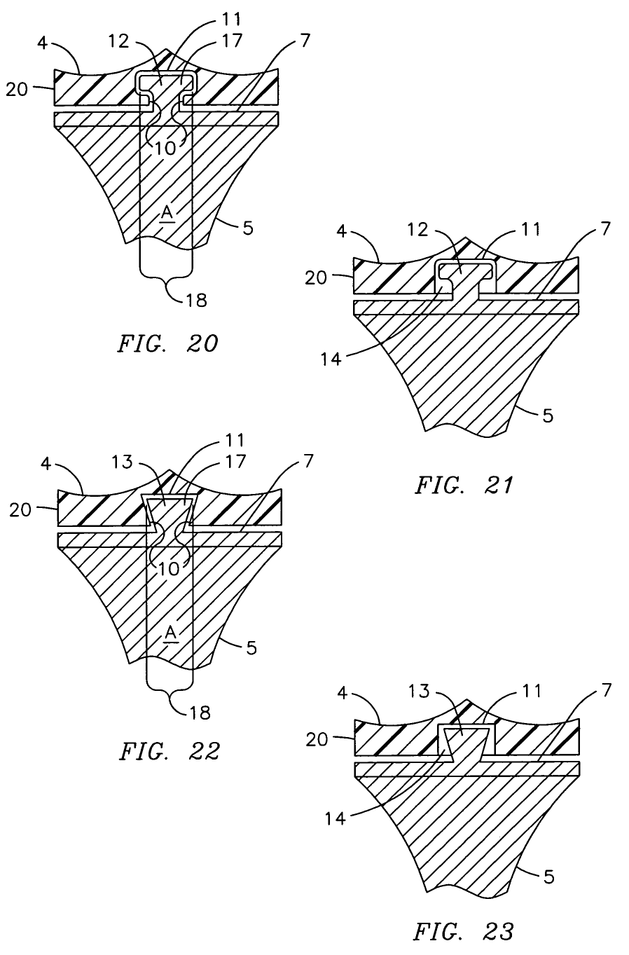

Next, in FIGS. 20 and 21, varying sectional views of a fifth embodiment of the tibial component and bearing of the present invention are shown. Similar to the fourth embodiment, the fifth embodiment has the extension 17 located on the tibial component 5 and the socket 9 located on the tibial bearing insert 20. However, the fifth embodiment has the extension 17 in the shape of a disc 12.

Finally, with reference to FIGS. 22 and 23, varying sectional views of a sixth embodiment of the tibial component and bearing of the present invention are shown. In the sixth embodiment, the extension 17 is in the shape of a trapezoid 13 and the central socket area 14 and track 11 are sized and shaped for acceptance of same.

Although the present invention is for tibial components and bearings of varying structures, the present invention is to be used as part of a total knee system for total knee arthroplasty. Thus, the bearing is conformable to the femoral condyles, femoral component, patellar implant and patella.

In addition, although only a limited number of shapes are shown as extensions and sockets, extensions and sockets of other shapes may also be utilized.

The use of the present invention will provide both axial rotation and anterior-posterior rotation of the prosthetic knee.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A knee joint prosthesis used in total knee arthroplasty comprising:
a tibial bearing insert having at least one extension;
said at least one extension is of a predetermined shape;
said at least one extension having a predetermined perimeter;
a tibial component having a tibial component base plate;
said tibial component having a predeterminedly shaped socket;
said socket having a central socket area that is similar in shape to the perimeter of the at least one extension wherein said at least one extension is insertable into said central socket area;
said tibial component having a predeterminedly shaped track within a base of the socket wherein said at least one extension is movable along and within said predeterminedly shaped track; and
said socket having at least one retaining edge that covers a predetermined portion of said track.

2. The knee joint prosthesis of claim 1 wherein:
said at least one extension shape is a sphere.

3. The knee joint prosthesis of claim 1 wherein:
said at least one extension shape is a disc.

4. The knee joint prosthesis of claim 1 wherein:
said at least one extension shape is a trapezoid.

5. The knee joint prosthesis of claim 1 wherein:
said at least one extension is secured to a bearing base plate secured to said tibial bearing insert.

6. The knee joint prosthesis of claim 2 wherein:
said at least one extension is secured to a bearing base plate secured to said tibial bearing insert.

7. The knee joint prosthesis of claim 3 wherein:
said at least one extension is secured to a bearing base plate secured to said tibial bearing insert.

8. The knee joint prosthesis of claim 4 wherein:
said at least one extension is secured to a bearing base plate secured to said tibial bearing insert.

9. A knee joint prosthesis used in total knee arthroplasty comprising:
a tibial component having at least one extension;
said at least one extension is of a predetermined shape;
said at least one extension having a predetermined perimeter;
a tibial bearing insert having a predeterminedly shaped socket;
said socket having a central socket area that is similar to the perimeter of the at least one extension wherein said at least one extension is insertable into said central socket area;
said tibial bearing insert having a predeterminedly shaped track within a base of the socket wherein said at least one extension is movable along and within said predeterminedly shaped track; and
said socket having at least one retaining edge that covers a predetermined portion of said track.

10. The knee joint prosthesis of claim 9 wherein:
said at least one extension shape is a sphere.

11. The knee joint prosthesis of claim 9 wherein:
said at least one extension shape is a disc.

12. The knee joint prosthesis of claim 9 wherein:
said at least one extension shape is a trapezoid.

13. The knee joint prosthesis of claim 9 wherein:
said at least one extension is secured to a tibial component base plate secured to said tibial component.

14. The knee joint prosthesis of claim 10 wherein:
said at least one extension is secured to a tibial component base plate secured to said tibial component.

15. The knee joint prosthesis of claim 11 wherein:
said at least one extension is secured to a tibial component base plate secured to said tibial component.

16. The knee joint prosthesis of claim 12 wherein:
said at least one extension is secured to a tibial component base plate secured to said tibial component.

17. The knee joint prosthesis of claim 1 wherein:
said at least one extension is metal; and
said tibial component base plate is metal.

18. The knee joint prosthesis of claim 1 wherein:
said at least one extension is polyethylene.

19. The knee joint prosthesis of claim 9 wherein:
said at least one extension is metal; and
said tibial component base plate is metal.

20. The knee joint prosthesis of claim 9 wherein:
said at least one extension is polyethylene.

21. A knee joint prosthesis used in total knee arthroplasty comprising:
a tibial bearing insert having a bearing base plate;
an extension secured to said bearing base plate;
said extension is a sphere;
said extension having a predetermined circumference;
a tibial component having a tibial component base plate;
said tibial component having a predeterminedly shaped socket;
said socket having a central socket area that is similar to the circumference of the extension wherein said at least one extension is insertable into said central socket area;
said tibial component having a predeterminedly shaped track within a base of the socket wherein said at least one extension is movable along and within said predeterminedly shaped track; and
said socket having at least one retaining edge that covers a predetermined portion of said track.
